(19) United States
(12) Patent Application Publication
(10) Pub. No.: US 2026/0315455 A1
(43) Pub. Date: Oct. 15, 2026

(54) ANTI-MICROBIAL SYNERGY OF ETHACRIDINE LACTATE PLUS ESSENTIAL OILS

(71) Applicant: Marie's Original Formulas LLC, Pearl River, NY (US)

(72) Inventor: Zalman Goodman, Monsey, NY (US)

(21) Appl. No.: 19/174,091

(22) Filed: Apr. 9, 2025

(51) Int. Cl. A61K 31/473; A61K 31/085; A61K 36/61; A61P 31/02

(52) U.S. Cl. CPC A61K 31/473; A61K 31/085; A61K 36/61; A61P 31/02

(57) ABSTRACT

Compositions and methods against bacterial infection. Compositions containing antibiotic and essential oil are demonstrated effective against multiply drug-resistant bacteria. Such compositions yield 100% kill rate against MRSA, P. aeruginosa and E. coliK1 . Antibiotic evaluated in studies include ethacridine lactate (“EL”). Essential oil evaluated in the studies include tea tree oil; which includes eugenol. The essential oil was pre-blended with an ointment base to form an ointment. While even 90%-100% ointment applied without EL to cultures of the bacteria does not produce 100% kill rates, EL in 95% ointment applied to the cultures does produce 100% kill rates, even at 0.2% EL (wt/wt). Expanded studies are to test other intercalating and non-intercalating antibiotics; essential oils; and ointment bases. Suture material and/or dressings with the compositions embedded, may transfer effective antimicrobial dosing to a patient, as would direct application of the compositions, either prophylactically or therapeutically.

### FIELD OF TECHNOLOGY

Aspects of the disclosure relate to treatment of bacterial infections. In particular, the disclosure provides compositions of treatments, and methods for treatment, that may be protective against infections caused even by bacterial strains resistant to multiple available antibiotics.

### BACKGROUND OF THE INVENTION

Increasing microbial resistance against previously efficacious antibiotic treatments is, in the words of a 2024 report of the Center for Disease Control and Prevention (“CDC”), “an urgent global public health threat.” Such resistance was associated with almost five million deaths worldwide in 2019 (study-data from before the bacterial resistance landscape's precipitous deterioration during and following the COVID pandemic). (Murray C J L, Ikuta K S, et al. (2022) Global burden of bacterial antimicrobial resistance in 2019: a systematic analysis. Lancet 399:629) A CDC 2019 report projected per annum over 2.8 million antibiotic-resistant bacterial infections in the United States, resulting in almost 36,000 deaths.

Despite improved awareness, reporting and treatment efforts, the societal toll of bacterial resistance is climbing. There has been a steady increase in the number of pathogenic bacterial strains becoming newly resistant not just to a small number of standard antibiotics but also becoming multiply drug-resistant (“MDR”) to a wide range of once highly potent but now largely ineffective available treatments. Reflecting this rise, the CDC 2019 U.S. annual numbers given above of severe infections and deaths ensuing directly from bacterial resistance represent a 40% and a 55% increase, respectively, over the 2013 numbers of about two million and about 23,000.

Not reflected in the CDC mortality statistics, but a cause for significant and growing concern, is the societal toll of serious, often debilitating and sometimes permanently so, sequalae of infections that patients survive.

Data underlying the statistics are, generally, aggregated from hospitals and other healthcare facilities, which provide the best resources for fighting such infections and preventing debilitation and death; those facilities also provide reliable records of failure of the resources. Because of the medical need to maintain an antiseptic environment for patient-protection and the widespread use of antibiotics to achieve such asepsis, those facilities may also be breeding grounds for hardy “survival-of-the-fittest” MDR bacterial strains that can severely challenge a patient's health.

A hospital/healthcare facility may serve as an optimal setting for potentially complicated births, wound/burn treatments, surgeries and other procedures requiring highly trained personnel and specialized resources. However, a bacterial infection contracted in such a setting may likely be one that the facility and staff, even with the best available medication and equipment at their disposal, may not be able to prevent from becoming life-threatening.

Three MDR bacterial strains that have gained notoriety in recent decades for their prevalence, lethality and/or target populations are methicillin-resistant Staphylococcus aureus (“MRSA”); Pseudomonas aeruginosa;and Escherichia coli K1. While these and other MDR bacterial strains may be susceptible to very powerful antibiotics, such as gentamicin and quinolones, typically those still effective antibiotics may be safely administered only sparingly and briefly to patients, and even then may carry significant risks of severe side effects.

Accordingly, it would be desirable to provide compositions, and methods of application of the same, that are both effective against MDR bacterial strains and safe for administration to the patient. Particularly urgent needs may exist for safe and effective anti-MDR treatments in healthcare's battles against MRSA, P. aeruginosa and E. coli K1.

2024 CDC reporting of 2019 data for the U.S. projected that MRSA is responsible for almost 324,000 in-hospital-and community-acquired cases, about 70,000 of those being severe infections requiring hospital care, with about 10,600 deaths per year. The resistant S. aureus responsible for MRSA, originally almost exclusively in-hospital in 2000, had already made the jump-presumably largely through prisons, barracks, migration centers and drug-needle reuse-to being broadly community-acquirable by 2015, along with widened resistance. Comparison of the 2019 data with the 2013 CDC data is complicated by the latter only including in-hospital-acquired cases, of which there were 80,000 cases and 11,000 deaths. While the death toll seems to have somewhat decreased, MRSA remains a persistent and deadly foe of human health.

MDR P. aeruginosa is responsible mainly for in-hospital-acquired cases, for which CDC 2017 data projected about 32,600 infections and about 2,700 deaths in the U.S. While currently having a lower casualty count nationally than MRSA, P. aeruginosa is considered a serious growing threat as it is resistant to a wider array of antibiotic molecules than MRSA. P. aeruginosa is known to cause serious post-surgery blood and/or urinary tract infections, as well as pneumonia and infections in other parts of the body. Another concern is that community-acquired P. aeruginosa cases, still relatively low in number, are increasing. With its broad immunity, there is a dearth of treatment options for MDR P. aeruginosa infection patients.

E. coli K1 is recognized as a highly virulent MDR pathogen responsible for life-long debilitation and death in target populations that society has historically protected, newborn babies and pregnant women (additionally targeted are the elderly and transplant recipients), often causing hospital-acquired meningitis that requires immediate emergency life-saving efforts. Recent advances in preventing and countering E. coli K1 infections may likely be offset by ever-growing successes in handling complicated pre-term and other high-risk deliveries yielding ever more vulnerable neonates and mothers. Additionally, vaccine programs and antibiotics aimed at other bacterial causes of neonatal meningitis, such as Neisseria meningitidis and Group B Streptococcus, have made headway in lowering the spread of meningitis from those bacteria. Although overall numbers may currently be unclear, some trends and percentages may serve to underscore the lethality and damage of E. coli K1. E. coli K1 has by now become among the most common causes of bacterial neonatal meningitis infection, with 30%-50% of cases leaving the newborns with neurologic complications of varying but usually profound severity, and 10%-15% killing the babies. While MDR E. coli K1 is still uncommon in adult community-acquired meningitis, the latter's rates of neurological complications of about 50% and of mortality of about 25% are causes of increasing concern.

A significant measure of evaluating the effectiveness of an antimicrobial against a bacterial strain is the rate at which that antimicrobial kills the given microbe under standard incubation conditions, the kill rate (or, inversely, the survival rate of the bacterial strain incubated with the antimicrobial). The more pathogenic a bacterial strain, the more important is the achievement of a 100% kill rate by the antimicrobial, since even a small number of bacteria left surviving, with minimal competition from other species—at, for instance, an antibiotic-cleansed surgical site—can readily multiply to overwhelm the patient.

Accordingly, it would also be desirable that the compositions provided, and methods of their application, achieve a 100% kill rate, as measured and confirmed by reliable, accurate laboratory testing. Given the prevalence, lethality and/or target populations of MRSA, P. aeruginosa and E. coli K1, those bacterial strains may represent significant target-subjects for laboratory testing of antimicrobial effectiveness of the provided compositions against infection.

A challenge failed by many antibiotic molecules in the face of bacterial acquisition of resistance, is maintaining high kill rates over years of use. The mechanisms of acquisition of resistance by bacteria exposed to a given antibiotic are varied, well understood and, usually, unavoidable. The search for new antibiotic molecules as alternatives to those with now-diminished kill rates, has often involved developing stronger antibiotics. As resistance is acquired against the successful alternatives, a spiraling war pitting bacteria against pharmaceutical scientists ensues, with the bacteria likely the eventual victors.

Accordingly, it would be desirable, also, that patient-safe compositions provided, and methods of their application, be effective against bacteria without inducing resistance. This may be achieved by utilizing:
a)components that have proven over decades (in some cases, centuries) to be patient-safe and continually effective;
b)in tandem, multiple molecular components with distinct anti-bacterial mechanisms; and
c)where those multiple anti-bacterial mechanisms are each unlikely to yet be thwarted by development of bacterial resistance.

Another challenge encountered in fighting bacteria on/in the patient's body is an ability of many bacteria to form biofilms by which the bacterial colony adheres to host tissue, cocooning itself away from direct contact with the patient's immune system and also from medicinal antimicrobial agents. MRSA, P. aeruginosa and E. coli K1 are among bacteria that form biofilms.

Accordingly, it would be desirable, also, that patient-safe compositions provided, and methods of their application, be effective in preventing the formation of biofilms and in degrading extant biofilms. This may be achieved by utilizing components that have demonstrated, well studied anti-biofilm properties.

### BRIEF DESCRIPTION OF THE DISCLOSURE

Compositions and methods for treatment of bacterial infections are provided that may be both effective against MDR bacterial strains and safe for administration to the patient.

The compositions and methods provided may avoid the pitfall of bacterial resistance by involving simultaneous administration of multiple antimicrobial components in multi-component compositions, in which the components have been safely used independently over decades without inducing immunity.

The safe compositions and methods provided may exhibit anti-biofilm properties, contributing to degradation of bacterial biofilms and prevention of their formation. Additionally, the safe compositions and methods provided may contribute to degradation of bacterial cell membranes and walls.

An unexpected and welcomed outcome of the use of the multi-component compositions of the invention was evidenced by third-party in vitro testing of the compositions against MRSA, P. aeruginosa and E. coli K1. For each of those MDR bacterial strains, the compositions of the invention achieved a 100% kill rate, as measured by the testing, at component concentrations that have well established human-use safety histories. At those same concentrations, the individual components did not achieve a 100% kill rate for any of those MDR pathogens.

Tea tree oil and eugenol, essential oil ingredients of the compositions and methods tested against the MDR bacterial strains, have been safely used clinically and have been studied for centuries in the context of various cultures' nutraceutical apothecaries, with a growing appreciation of late in Western medicine. (Carson C F, Hammer K A, et al. (2006) Melaleuca alternifolia (Tea Tree) oil: a review of antimicrobial and other medicinal properties. Clin Microbiol Rev. 19:50; Marchese A, Barbieri R, et al. (2017) Antimicrobial activity of eugenol and essential oils containing eugenol: A mechanistic viewpoint. Crit Rev Microbiol. 43:668) Over the centuries of their use, there has been no indication of bacterial resistance developing against the essential oils.

Ethacridine lactate, an antibiotic ingredient of the compositions and methods tested against the MDR bacterial strains, has been safely used clinically and has been studied for over a century. Low concentration ethacridine lactate has been utilized historically as a useful disinfectant, with its intercalating antibiotic and immunoregulatory properties becoming appreciated in recent decades. (Kukowska M (2017) Amino acid or peptide conjugates of acridine/acridone and quinoline/quinolone-containing drugs. A critical examination of their clinical effectiveness within a twenty-year timeframe in antitumor chemotherapy and treatment of infectious diseases. European Journal of Pharmaceutical Sciences 109:587) Over the century of its use, there has been no indication of bacterial resistance developing against ethacridine lactate.

A typical delivery form of ethacridine lactate used in wound treatment is as 0.1% (wt/wt) solution, such as Rivinol®, available from CHEMAX PHARMA, Sofia, Bulgaria. Another delivery form of ethacridine lactate used in wound treatment is as dry yellow crystalline powder, such as that used, mixed with other ingredients, in ActXT Powder, developed by Maries Originals®, Pearl River, NY.

Bakhtar (Bakhtar B (2013) Amazing Topical Protocol and Novel Powder Dressing Combination Formula in Treatment of Diabetic Foot Wounds. Iran J Public Health 42:1327) alleges good results in treatment of diabetic ulcer a) by cleansing the wound with 0.1% ethacridine lactate solution and in combination with metronidazole, in normal saline; and b) by use of a “powder combination of ethacridine lactate (0.1 %), zinc oxide, oak fruit and ginkgo biloba” that was “sprinkled on wound” BID. The first Bakhtar result is unremarkable, as successful disinfectant use of low concentration ethacridine lactate solutions on open wounds has been well known since before 1920. The second Bakhtar result also is expected from well known properties of zinc oxide and of low concentration ethacridine lactate. Bakhtar does not teach, explicitly or implicitly, this disclosure's novel principle of potent anti-microbial synergy achieved by combinations of ethacridine lactate and essential oils against bacterial infection.

The methods may include methods of applying the compositions. Application of the compositions may include therapeutic application against extant bacterial infection. The therapeutic application may include topical application of the compositions to a treatment site. The treatment site may include the extant bacterial infection. The therapeutic application may include application via wound closure material containing the compositions. The therapeutic application may include application via surgical sutures containing the compositions. The therapeutic application may include application via dressings containing the compositions.

Application of the compositions may include prophylactic application configured to preclude bacterial infection. The prophylactic application may include topical application of the compositions to a treatment site. The prophylactic application may include application via wound closure material containing the compositions. The prophylactic application may include application via surgical sutures containing the compositions. The prophylactic application may include application via dressings containing the compositions.

The wound closure material may be applied to the treatment site. The surgical sutures may be applied to the treatment site. The dressings may be applied to the treatment site. The dressings may include bandages.

### BRIEF DESCRIPTION OF THE DRAWINGS

The objects and advantages of the invention will be apparent upon consideration of the following detailed description, taken in conjunction with the accompanying drawings, in which like reference characters refer to like parts throughout, and in which:

FIG. 1 shows tables of analytical testing results of effectiveness of composition components and of multi-component compositions formulated in accordance with principles of the invention;

FIG. 2 shows bar graphs representing results of testing MRSA in accordance with principles of the invention;

FIG. 3 shows bar graphs representing results of testing P. aeruginosa in accordance with principles of the invention; and

FIG. 4 shows bar graphs representing results of testing E. coli K1 in accordance with principles of the invention.

### DETAILED DESCRIPTION OF THE DISCLOSURE

Compositions and methods for application to a treatment site are provided. The compositions and methods may be used against bacterial infection. The compositions and methods may be used against bacterial infection at the treatment site. The compositions may be antimicrobial compositions.

The compositions and methods may be used to prevent bacterial infection at the treatment site. The treatment site may include a site selected for application of the compositions to preclude a bacterial infection.

The treatment site may include a bacterial infection. The compositions may be applied to the bacterial infection. The compositions may be applied, according to the methods, to the bacterial infection. The compositions and methods may be used to treat the bacterial infection. The compositions and methods may be used to reduce the bacterial infection. The compositions and methods may be used to kill bacteria of the bacterial infection.

The compositions may be used to perform steps of the methods. The methods may include administration of the compositions to the treatment site.

The compositions may include, and the methods may involve, compositions of matter for therapeutic application to the treatment site. The compositions may include, and the methods may involve, the compositions of matter for prophylactic application to the treatment site.

The compositions may include a composition. The compositions may include a single composition. The term “composition” may include one or more than one composition.

The composition may include antibiotics. The term “antibiotic” may include one or more than one antibiotic.

The composition may include essential oils. The term “essential oil” may include one or more than one essential oil.

The composition may include an antibiotic first component. The composition may include an essential oil second component. The composition may include both the antibiotic first component and the essential oil second component. The composition may include both the antibiotic first component and the essential oil second component compounded together.

The composition may include, and the methods may involve, both the antibiotic first component and the essential oil second component administered together to a treatment site. The composition may include, and the methods may involve, the antibiotic first component and the essential oil second component administered separately to the treatment site. The composition may include, and the methods may involve, administering the antibiotic first component to the treatment site and then administering the essential oil second component to the treatment site. The composition may include, and the methods may involve, administering the antibiotic first component to the treatment site and then overlaying the antibiotic first component with the essential oil second component.

The composition may include, and the methods may involve, administering the essential oil second component to the treatment site (preferably via an ointment) and then administering the antibiotic first component to the treatment site. The composition may include, and the methods may involve, administering the essential oil second component to the treatment site and then overlaying the essential oil second component with the antibiotic first component. The composition may include, and the methods may involve, administering the essential oil second component to the treatment site via an ointment and then overlaying the essential oil second component-containing ointment with the antibiotic first component in powder form.

When the antibiotic first component is incubated under standardized conditions, at a non-zero concentration but absent the essential oil second component, with a population of viable pathogenic bacteria of at least about a million cells, a non-zero population of the viable pathogenic bacteria may be left. The antibiotic first component at its concentration, without the essential oil second component, may not attain a 100% kill rate of the pathogenic bacteria.

The standardized conditions may include nutrient medium, such as RPMI cell culture medium. The RPMI may be supplemented with LB medium. The standardized conditions may include nutrient plating agar for colony forming units (“cfu”) culturing and counting. The standardized conditions may include an incubation temperature. The incubation temperature may be about 37 °C. The standardized conditions may include a monitored number of hours of incubation in RPMI and also a monitored number of hours of incubation on agar.

Standard laboratory analytical assay methods may provide population counts before and after the cell incubation. The standard laboratory analytical assay methods may include optical density measurements of solutions/suspensions of cells in nutrient medium. The standard laboratory analytical methods may include counting of cfu that may have grown on the nutrient agar, providing a count of cells that survived the pre-plating incubation in nutrient medium.

The standard laboratory analytical assay methods may provide population counts before and after cell incubation with the antibiotic.

When the essential oil second component is incubated under the standardized conditions, at a non-zero concentration but absent the antibiotic first component, with a population of the viable pathogenic bacteria of at least about a million cells, a non-zero population of the viable pathogenic bacteria may be left. The essential oil second component at its concentration, without the antibiotic first component, may not attain a 100% kill rate of the pathogenic bacteria. The standard laboratory analytical assay methods may provide population counts before and after cell incubation with the essential oil.

When the composition, with a combination of both the first component at the antibiotic concentration and the second component at the essential oil concentration, is incubated under the standardized conditions with a population of the viable pathogenic bacteria of at least about a million cells, a population of zero of the viable pathogenic bacteria may be left. The composition, with a combination of both the first component at the antibiotic concentration and the second component at the essential oil concentration, may attain a 100% kill rate of the pathogenic bacteria. The standard laboratory analytical assay methods may provide population counts before and after cell incubation with the combination of the antibiotic and the essential oil.

A positive control and a negative control may be run alongside testing of the combined and separate components of the compositions. The positive control may include incubating a population of the viable pathogenic bacteria of at least about a million cells, with a powerful antibiotic, such as gentamicin (or some other antibiotic not being tested) with no essential oil, at a concentration expected to typically attain a 100% kill rate. The negative control may include incubating a population of the viable pathogenic bacteria of at least about a million cells, with neither the antibiotic nor the essential oil. The standardized conditions employed for testing the populations incubated with both or only one of the antibiotic and the essential oil, may be employed for the positive and negative controls. The standard laboratory analytical assay methods employed for quantifying the populations incubated with both or only one of the antibiotic and the essential oil, may be employed for the positive and negative controls.

The compositions' combined components leaving a population of zero of the viable pathogenic bacteria may indicate an antimicrobial synergy attained by the combination of the antibiotic and the essential oil at their respective concentrations. The synergy of the combination may yield greater lethality toward the pathogenic bacteria than separately exhibited by either the antibiotic or the essential oil, at the respective concentrations. The synergy of the combination may yield greater lethality toward the pathogenic bacteria than the sum of the lethalities separately exhibited by either the antibiotic or the essential oil, at the respective concentrations.

The pathogenic bacteria may include a multiply drug-resistant (MDR) species. The pathogenic bacteria may include Gram positive methicillin-resistant Staphylococcus aureus (MRSA). The compositions and methods may be used against a MRSA infection. The compositions may be applied to the treatment site against the MRSA infection. The compositions may be applied, according to the methods, to the treatment site against the MRSA infection. The compositions and methods may be used prophylactically to prevent the MRSA infection. The compositions and methods may be used therapeutically against the MRSA infection.

The antibiotic first component, when incubated under the standardized conditions, at a non-zero antibiotic concentration but absent the essential oil second component, with a population of live MRSA bacteria of at least about a million cells, may leave, for instance, more than about 0.3% of the population viable. The intentionally underestimated value of 0.3% viable population was determined, by the standard analytical laboratory assay methods, in a series of tests that yielded an average of about 3.75% surviving of an initial about one million MRSA cells incubated with the highest antibiotic concentration tested (which was about five-fold the antibiotic concentration envisioned for commercial use). An extrapolation an order of magnitude below any actual percentage experimentally encountered of the surviving MRSA population, against the highest antibiotic concentration tested, may be made by taking 10% of the 3.75% average result, and truncating the product to its first non-zero digit. Higher survivability percentages of MRSA cells than 3.75% were obtained against lower concentrations of antibiotic.

The antibiotic first component, when incubated under the standardized conditions, at a non-zero antibiotic concentration but absent the essential oil second component, with a population of live MRSA bacteria of at least about a million cells, may leave a non-zero population of, for instance, more than about 3000 viable MRSA cells. The intentionally underestimated value of more than about 3000 viable cells was determined, by the standard analytical laboratory assay methods, in a series of tests that yielded an average cell count of about 37,500 surviving out of an initial ~10⁶ MRSA cells incubated with the highest antibiotic concentration tested (about five-fold the antibiotic concentration envisioned for commercial use). An extrapolation an order of magnitude below any actual number experimentally encountered of the surviving MRSA cell count, against the highest antibiotic concentration tested, may be made by taking 10% of the 37,500 cell count average result, and rounding the product down to its lowest whole number of thousands. Higher counts of surviving MRSA cells than 37,500 out of 10⁶ were obtained against lower concentrations of antibiotic The essential oil second component, when incubated under the standardized conditions, at a non-zero essential oil concentration but absent the antibiotic first component, with a multi-million cell population of live MRSA bacteria of at least about a million cells, may leave, for instance, more than about 1% of the population viable, as determined by the standard analytical laboratory assay methods. The intentionally underestimated value of 1% viable population was determined, by the standard analytical laboratory assay methods, in a series of tests that yielded an average of about 15% surviving of an initial about one million MRSA cells incubated with the highest essential oil concentration tested (which was slightly higher than the essential oil concentration envisioned for commercial use). An extrapolation an order of magnitude below any actual percentage experimentally encountered of the surviving MRSA population, against the highest essential oil concentration tested, may be made by taking 10% of the 15% average result, and truncating the product to its first non-zero digit. Higher survivability percentages of MRSA cells than 15% were obtained against lower concentrations of essential oil.

The essential oil second component, when incubated under the standardized conditions, at a non-zero essential oil concentration but absent the antibiotic first component, with a population of live MRSA bacteria of at least about a million cells, may leave a non-zero population of, for instance, more than about 1×10⁴ viable MRSA cells. The intentionally underestimated value of more than about 1×10⁴ viable cells was determined, by the standard analytical laboratory assay methods, in a series of tests that yielded an average cell count of about 150,000 surviving out of an initial ~10⁶ MRSA cells incubated with the highest essential oil concentration tested (slightly higher than the essential oil concentration envisioned for commercial use). An extrapolation an order of magnitude below any actual number experimentally encountered of the surviving MRSA cell count, against the highest essential oil concentration tested, may be made by taking 10% of the 150,000 cell count average result, and rounding the product down to its lowest whole number of ten-thousands. Higher counts of surviving MRSA cells than 150,000 out of a million were obtained against lower concentrations of essential oil.

The composition, with a combination of the first component at the antibiotic concentration and the second component at the essential oil concentration, incubated under the standardized conditions with a population of live MRSA bacteria of at least about a million cells, may leave 0% (zero) of the MRSA cells viable, as determined by the standard laboratory assay methods. This zero % result may demonstrate a synergistically elevated MRSA kill rate of the combined components of the composition over the sum of individual MRSA kill rates of the antibiotic first component and of the essential oil second component.

Positive and negative controls may be run for each set of initial viable MRSA cell populations. The positive controls, run with gentamicin and no essential oil, may yield populations of zero viable MRSA cells. The negative controls, run with neither the antibiotic nor the essential oil, may yield viable MRSA populations of greater cell counts than the initial counts of at least about a million. Such greater cell counts of viable MRSA populations of the negative controls, may include MRSA populations of greater than about 3.0×10⁶ live MRSA bacteria. Such greater cell counts of viable MRSA populations of the negative controls, may include any size MRSA populations greater than the initial count of at least about a million. Any size MRSA population greater than the initial count of the negative controls, may include MRSA populations of greater than about 1.0×10⁷ live MRSA bacteria. A cell count of viable MRSA populations of the negative controls, may include a MRSA population of less than the initial population count.

The pathogenic bacteria may include Gram negative neuropathogenic Escherichia coli K1 (E. coli K1). The pathogenic bacteria may include Gram negative Pseudomonas aeruginosa(P. aeruginosa). The pathogenic bacteria may include any other pathogenic bacteria species or strain. Any other pathogenic bacteria species or strain may include Gram positive Streptococcus pyogenes(S. pyogenes). S. pyogenes may be responsible hundreds of millions of infections worldwide, mostly non-fatal, but may be a leading cause of septic-shock deaths among pregnant and postpartum women.

The antibiotic component may include an intercalating agent. The intercalating agent may intercalate between bases of bacterial DNA. The intercalating agent may frameshift a section of the bacterial DNA into which the agent intercalates. The intercalating agent may be disruptive of bacterial cellular functions. The intercalating agent may be disruptive of bacterial DNA transcription. The intercalating agent may be disruptive of bacterial DNA replication.

The intercalating agent may include an acridine. Acridine is known to cause DNA frameshifts. The intercalating agent may include ethacridine lactate (EL). The intercalating agent may include a fluoroquinolone. The fluoroquinolone may include ciprofloxacin. The fluoroquinolone may include moxifloxacin. The intercalating agent may include a topoisomerase inhibitor. The topoisomerase inhibitor may include gepotidacin. The intercalating agent may include any other class of intercalating agent. Any other class of intercalating agent may include a spiropyrimidinetrione. The spiropyrimidinetrione may include zoliflodacin.

Testing results reported herein on effects on bacterial cultures of composition components separately or combined, deal with EL as the antibiotic first component. The reported results supporting synergy of lethality of the composition's combined components, have validity regardless of understanding underlying mechanisms of the synergy. Nevertheless, it may be edifying to explore possible underlying mechanisms.

EL has served as an antimicrobial antiseptic for over a century, without evidence of bacterial resistance having developed against it. The dearth of bacterial resistance to EL may be due to EL being historically limited to human use and at low concentrations, giving bacteria low exposure to EL. The dearth of bacterial resistance to EL may be due to EL serving as an intercalating agent. Bacterial resistance to specific intercalating-agent antibiotics has been reported. Resistance to specific intercalating-agent antibiotics seems to be through the bacteria thwarting the transport of intercalating agents into the bacterial cell, enhancing their transport out of the bacterial cell, or presenting mutated conformations of bacterial cell molecules associated with intercalation processes.

Several antibiotics, including intercalating antibiotics such as some fluoroquinolones, may have severe side effects in humans with long-term administration or at high concentrations and are now usually prescribed only selectively at low-concentration doses and, in specific cases, mainly topically. Additionally, unlike accepted practices for early-generation antibiotics of the 1950s-1970s, such as penicillins' widespread administration to farm animals, powerful later-generation antibiotics are generally not used outside of human administration. With lower exposure, such antibiotics may regain their low-resistance profiles as non-mutated bacterial populations outcompete and “demographically” overwhelm the mutated subpopulations. Such low-exposure antibiotics may become safe for human administration without inducing resistance. Such antibiotics, at very low concentrations, may be safe for human administration but not effective on their own to attain 100% kill rates. It is envisioned that, besides EL, other low-concentration/low-resistance antibiotics may exhibit synergy of lethality when combined with essential oil. Other low-concentration/low-resistance antibiotics may include intercalating agents.

Essential oils have been implicated in the degradation of bacterial biofilms, cell walls and cell membranes. (Martínez A, Manrique-Moreno M, et al. (2021) Effect of Essential Oils on Growth Inhibition, Biofilm Formation and Membrane Integrity of Escherichia coli and Staphylococcus aureus. Antibiotics 10:1474; Andrade-Ochoa S, Chacón-Vargas K F, et al. (2021) Differential Antimicrobial Effect of Essential Oils and Their Main Components: Insights Based on the Cell Membrane and External Structure. Membranes 11:405) The essential oil component of the composition may contribute to degradation of bacterial biofilms, cell walls and cell membranes. Such degradation may facilitate increased exposure of the bacterial cells to the antibiotic component of the compositions, increased diffusion of the antibiotic into the cells, and/or decreased transport of the antibiotic out of the cells. It is envisioned that, besides essential oils tested as reported herein, other essential oils may exhibit synergy of lethality when combined with antibiotic. Other essential oils may include oils of rosemary, of thyme or any other suitable antimicrobial essential oil. Any other suitable antimicrobial essential oil may include cinnamon oil or cinnamon bark oil. Any other suitable antimicrobial essential oil may include oil of oregano. Any of the essential oils described herein, or any other suitable essential oils, should be considered as essentials oils for the purpose, and within the scope, of this patent application.

The composition may include EL. The antibiotic concentration incubated with the populations of the viable pathogenic bacteria may include about 0.01% to about 1.0% (wt/wt) EL. The antibiotic concentration incubated with the populations of the viable pathogenic bacteria may include about 0.2% (wt/wt) EL. The antibiotic concentration incubated with the populations of the viable pathogenic bacteria may include any other suitable concentration of EL. Any other suitable antibiotic concentration of EL for incubation with the populations of the viable pathogenic bacteria may include about 0.1% (wt/wt) EL. Any other suitable antibiotic concentration of EL for incubation with the populations of the viable pathogenic bacteria may include about 1.5% (wt/wt) EL. Any other suitable antibiotic concentration of EL antibiotic concentration for incubation with the populations of the viable pathogenic bacteria may be included in a range of about 0.01% (wt/wt) EL to about 2.5% (wt/wt) EL.

The composition may include essential oil. The essential oil may be at any essential oil concentration. The essential oil may be at an essential oil concentration not exceeding about 90% (vol/wt). The essential oil may be at an essential oil concentration not exceeding about 75% (vol/wt). The essential oil may be at an essential oil concentration not exceeding about 50% (vol/wt). The essential oil may be at an essential oil concentration not exceeding about 40% (vol/wt). The essential oil may be at an essential oil concentration not exceeding about 30% (vol/wt). The essential oil may be at an essential oil concentration not exceeding about 20% (vol/wt). The essential oil may be at an essential oil concentration not exceeding about 10% (vol/wt). The essential oil may be at an essential oil concentration not exceeding about 5% (vol/wt). The essential oil may be at an essential oil concentration included in a range of about 2.5% (vol/wt) essential oil to about 95% (vol/wt) essential oil.

The composition may include eugenol. The essential oil concentration incubated with the populations of the viable pathogenic bacteria may include about 0.05%-5.0% (vol/wt) eugenol. The essential oil concentration incubated with the populations of the viable pathogenic bacteria may include about 0.3% (vol/wt) eugenol. The essential oil concentration incubated with the populations of the viable pathogenic bacteria may include any other suitable concentration of eugenol. Any other suitable antibiotic concentration of eugenol for incubation with the populations of the viable pathogenic bacteria may include about 0.02% (vol/wt) eugenol. Any other suitable antibiotic concentration of eugenol for incubation with the populations of the viable pathogenic bacteria may include about 6.5% (vol/wt) eugenol. Any other suitable antibiotic concentration of eugenol for incubation with the populations of the viable pathogenic bacteria may be included in a range of about 0.01% (vol/wt) eugenol to about 15% (vol/wt) eugenol.

The composition may include tea tree oil. The essential oil concentration incubated with the populations of the viable pathogenic bacteria may include about 0.1%-10.0% (wt/wt) tea tree oil. The essential oil concentration incubated with the populations of the viable pathogenic bacteria may include about 4.0% (wt/wt) tea tree oil. The essential oil concentration incubated with the populations of the viable pathogenic bacteria may include any other suitable concentration of tea tree oil. Any other suitable antibiotic concentration of tea tree oil for incubation with the populations of the viable pathogenic bacteria may include about 0.05% (wt/wt) tea tree oil. Any other suitable antibiotic concentration of tea tree oil for incubation with the populations of the viable pathogenic bacteria may include about 12% (wt/wt) tea tree oil. Any other suitable antibiotic concentration of tea tree oil for incubation with the populations of the viable pathogenic bacteria may be included in a range of about 0.01% (vol/wt) tea tree oil to about 20% (vol/wt) tea tree oil.

The essential oil component may be blended, prior to incubation with the pathogenic bacteria, with an ointment base. The antibiotic component may be blended, prior to incubation with the pathogenic bacteria, with the ointment base. The composition of the combination of the essential oil and the antibiotic may be blended, prior to incubation with the pathogenic bacteria, with the ointment base. The ointment base may include any suitable ointment base. Any suitable ointment base may include jojoba oil. The ointment base may include about 1%-15% (wt/wt) jojoba oil.

The ointment may include zinc oxide. The ointment may include Zn²⁺. The ointment may include zinc as a drying agent. The ointment may include zinc as an anti-fungal.

The composition may include zinc oxide. The composition may include Zn²⁺. The composition may include zinc as a drying agent. The composition may include zinc as an anti-fungal. The composition may include zinc at less than about 10% (wt/wt). The composition may include zinc at less than about 5% (wt/wt). The composition may include zinc at less than about 2.5% (wt/wt). The composition may include zinc at less than about 1% (wt/wt).

The essential oil may be applied to the treatment site via an ointment base. The antibiotic may be applied to the treatment site via the ointment base. The composition of the combination of the essential oil and the antibiotic may be applied to the treatment site via the ointment base. The composition of the combination of the essential oil and the antibiotic may be applied, within the ointment base, to the treatment site. The composition of the combination of the essential oil and the antibiotic may be applied to the treatment site, within an ointment that includes the combination and the ointment base.

An ointment base into which the composition was blended, may be embedded within a dressing. The dressing may be a prepared dressing. Such composition-containing dressing may be configured to transfer a dose of the composition's combination to a subject dressed with the composition-containing dressing. A method of administering the composition may include applying the composition-containing dressing to the treatment site.

An ointment base into which the composition was blended, may be embedded within a wound closure material. Such composition-containing wound closure material may be configured to transfer a dose of the composition's combination to a wound closed with the wound closure material. A method of administering the composition may include closing a wound with the composition-containing wound closure material.

An ointment base into which the composition was blended, may be embedded within a suture material. Such composition-containing suture material may be configured to transfer a dose of the composition's combination to a wound closure sutured with the suture material. A method of administering the composition may include suturing a wound with the composition-containing suture material.

The antibiotic may be applied in liquid form to the treatment site. The essential oil may be applied in liquid form to the treatment site. The combination of the essential oil and the antibiotic may be applied in liquid form to the treatment site. The combination of the essential oil and the antibiotic may be applied, within a liquid, to the treatment site.

The antibiotic may be applied to the treatment site via a liquid. The essential oil may be applied to the treatment site via a liquid. The composition of the combination of the essential oil and the antibiotic may be applied in liquid form to the treatment site. The composition of the combination of the essential oil and the antibiotic may be applied, within the liquid, to the treatment site.

A liquid containing the composition may be embedded within a dressing that may be applied to the treatment site. Such composition-wetted dressing may be configured to transfer a dose of the composition's combination to a subject dressed with the composition-wetted.

A liquid containing the composition may be embedded within a suture material that may be applied to the treatment site. Such composition-wetted suture material may be configured to transfer a dose of the composition's combination to a wound closure sutured with the suture material.

Compositions and methods of their application as described herein are illustrative. Some compositions and methods in accordance with the invention will now be described in connection with the FIGs. The FIGs present results of accredited third party laboratory testing. The testing was conducted to quantify antimicrobial activity of components of the compositions and of the multi-component compositions in accordance with the principles of the invention. The testing was run, against each of MRSA, P. aeruginosa and E. coli K1, on:
a) essential oils (in ointment base) without EL;
b) EL without essential oils; and
c) EL plus essential oils (in ointment base).

Overall protocol: Known cell-count aliquots of each bacterial strain for each test-set were incubated with negative and positive controls or with each of a)-c) above; then serially diluted for plating and overnight incubation; followed by colony counting. Multiple replicates were run of each experiment. Values given in the FIGs are averages over the replicates. The bars of the graphs feature upper error-limits, indicating ranges of values obtained above the averages depicted by the bars.

Detailed protocol (with all cfu counts and all concentrations, volumes, times, temperatures, reported within standard lab practice deviations): Concentrations of initial bacterial cultures grown overnight in standard commercial growth medium (RPMI and/or LB) were adjusted, as measured by optical density, to 1×10⁸ colony forming units (“cfu”) per ml. Aliquots of 10 microliter of those cultures, each containing 1×10⁶ cfu, were incubated for two hours at 37 °C alone (as negative experimental control), or with gentamicin at 100 μg/mL concentration (as positive experimental control, with expected 100% kill rate), or with the indicated amounts of a)essential oil ointment without EL, the ointment diluted by RPMI/LB as necessary to achieve each indicated ointment amount; or b)EL without essential oil, the EL diluted by RPMI/LB as necessary to achieve each indicated EL amount; or c)the multi-component composition of EL plus essential oils, at 95% ointment (EL and RPMI/LB constituting the remaining 5%). After the two-hour incubation, serial 1:10 dilutions were performed on the negative control and the positive control and on all composition-containing samples. The multiple dilutions washed out gentamicin, composition components and ointment base, and stepwise reduced the population counts to allow for post-plating cfu counting. Those precision-diluted test samples were plated onto nutrient agar, followed by overnight incubation at 37 °C and then by counting of viable colonies, to give counts of the bacterial cell populations immediately following the two-hour incubations and then growing overnight.

Ointment used in the experiments included a blend of essential oils not exceeding about 75% (vol/wt), with the essential oils including eugenol and tree oil, in a jojoba oil ointment base. The ointment included zinc at about 0.67% (wt/wt).

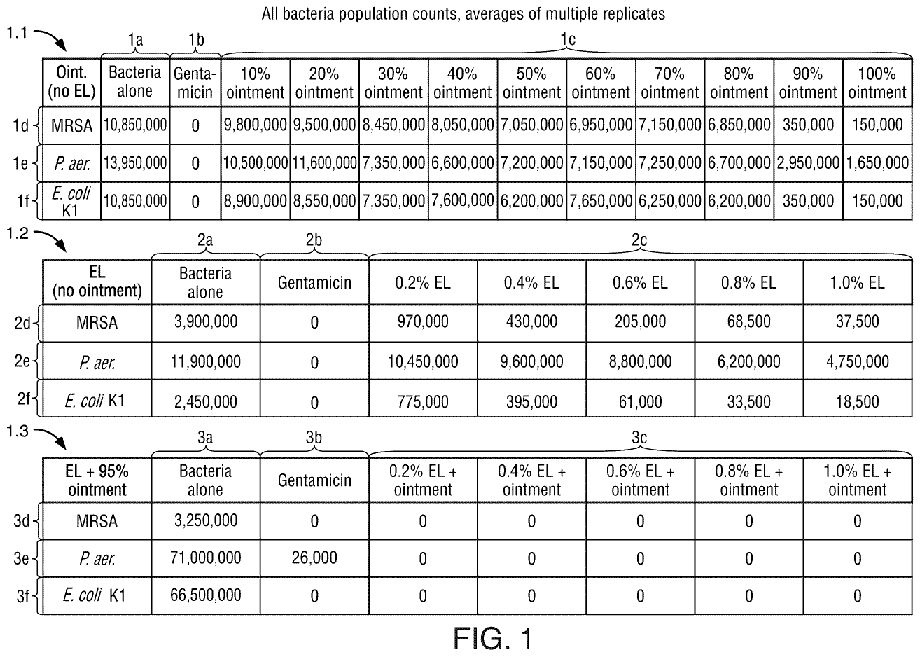

FIG. 1 shows three Tables (1.1, 1.2, 1.3) of testing results, giving bacterial population counts.

Table 1.1 presents bacterial cell counts of bacterial cells arrayed in column:
(1a) with no antimicrobial composition components (negative control);
(1b) against gentamicin (positive control); and
(1c) against indicated percentages of ointment without EL.

Table 1.1 presents the results in row:
(1d) for MRSA;
(1e) for P. aeruginosa;and
(1 f) for E. coli K1.

It should be noted that the 90% ointment result represents a percentage of bacteria surviving relative to the no-antibiotic (negative control) result reported in column 1a, of about 3.2% for MRSA; about 21.1% for P. aeruginosa;and about 3.2% for E. coli K1. At 100% ointment, the surviving percentage is about 1.3% for MRSA; about 11.8% for P. aeruginosa;and about 1.4% for E. coli K1. Interpolation to likely values at 95% ointment—the ointment concentration used in testing the multi-component composition—yields about 2.3% for MRSA; about 16.5% for P. aeruginosa;and about 2.3% for E. coli K1.

Table 1.2 presents bacterial cell counts of bacterial cells arrayed in column:
(2a) with no antimicrobial composition components (negative control);
(2b) against gentamicin (positive control); and
(2c) against indicated percentages of EL without ointment.

Table 1.2 presents the results in row:
(2d) for MRSA;
(2 e) for P. Aeruginosa;and
(2 f) for E. Coli K1.

It should be noted that the 0.2% EL result represents a percentage of bacteria surviving relative to the no-antibiotic (negative control) result reported in column 2a, of about 24.9% for MRSA; about 87.8% for P. aeruginosa;and about 31.6% for E. coli K1. Surviving percentages diminish as % EL increases, with 1.0% EL results being about 1.0% for MRSA; about 39.9% for P. aeruginosa;and about 0.8% for E. coli K1.

Table 1.3 presents bacterial cell counts of bacterial cells arrayed in column:
(3a) with no antimicrobial composition components (negative control);
(3b) against gentamicin (positive control); and
(3 c) against 95% ointment bearing essential oil plus indicated percentages of EL in accordance with the invention.

Table 1.3 presents the results in row:
(3d) for MRSA;
(3e) for P. Aeruginosa;and
(3f) for E. coli K1.

It should be noted that all tested percentages of EL-plus-ointment yielded a 0% bacterial survivability rate—a 100% kill rate—for each of MRSA, P. aeruginosa and E. coli K1. Particularly at low percentages of EL-plus-ointment compositions, these results evidence a potent synergy of lethality of the multi-component composition over lethalities exhibited by the composition's individual components.

Given a general consideration of using as low an antibiotic dose as possible to effect 100% kill rate, about 0.20% EL may be a dose of choice for an EL-plus-ointment therapeutic/prophylactic product. EL has been safely used as a disinfectant for over a century at about 0.10%. EL has been safely used topically for years at about 0.20%, without patient-or clinician-reports of safety issues. In vitro cytotoxicity studies on human cells have consistently demonstrated a generally considered acceptable sub-20% cellular death in 0.2% EL-plus-ointment, with an average approximately 25% cellular death in 0.5% EL-plus-ointment.

In administration to a treatment site, a multi-component composition of antibiotic first ingredient and essential oil second ingredient may include less than about 0.5% EL. In administration to a treatment site, the multi-component composition of antibiotic first ingredient and essential oil second ingredient may include less than about 0.25% EL. In administration to a treatment site, the multi-component composition of antibiotic first ingredient and essential oil second ingredient may include less than about 0.1% EL.

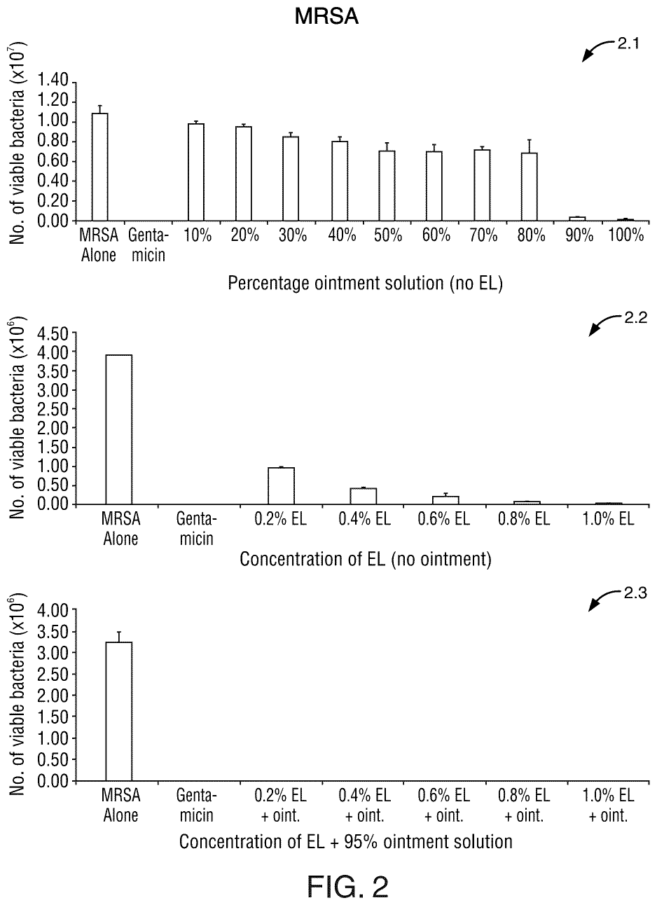

FIG. 2 shows three bar graphs (2.1, 2.2, 2.3) of test results against MRSA, portraying in graphical representation the MRSA data given in the Tables of FIG. 1.

Graph 2.1 portrays results tabulated in row 1d of Table 1.1. Graph 2.2 portrays results tabulated in row 2d of Table 1.2. Graph 2.3 portrays results tabulated in row 3d of Table 1.3.

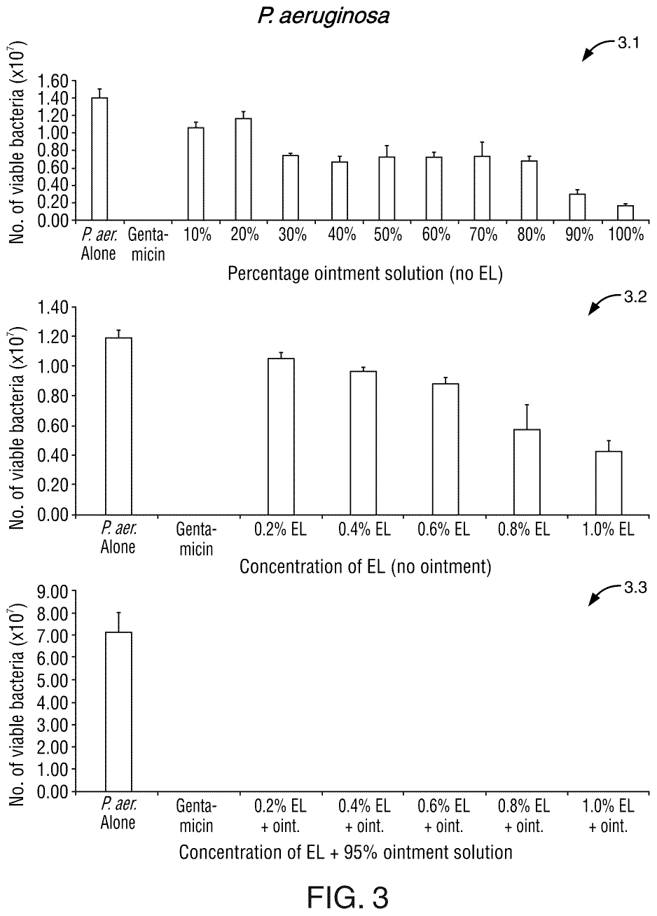

FIG. 3 shows three bar graphs (3.1, 3.2, 3.3) of test results against P. aeruginosa, portraying in graphical representation the P. aeruginosa data given in the Tables of FIG. 1.

Graph 3.1 portrays results tabulated in row 1e of Table 1.1. Graph 3.2 portrays results tabulated in row 2e of Table 1.2. Graph 3.3 portrays results tabulated in row 3e of Table 1.3.

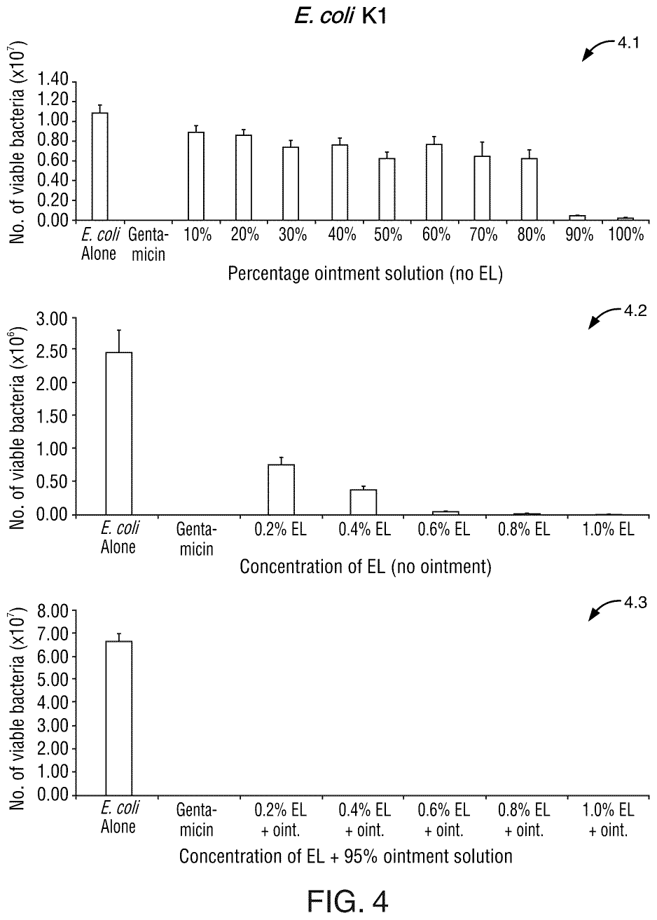

FIG. 4 shows three bar graphs (4.1, 4.2, 4.3) of test results against E. coli K1, portraying in graphical representation the E. coli K1 data given in the Tables of FIG. 1.

Graph 4.1 portrays results tabulated in row 1f of Table 1.1. Graph 4.2 portrays results tabulated in row 2f of Table 1.2. Graph 4.3 portrays results tabulated in row 3f of Table 1.3.

Expanded studies are continuing and further studies are planned, focusing on use of the compositions and methods disclosed herein against bacterial infection. Additionally envisioned are studies involving different antibiotics. The antibiotics may include intercalating agents. The studies may involve different essential oils. The studies may involve different relative amounts of essential oils with and without ointment base. The studies may involve different ointment bases.

All references cited in the present application are incorporated in their entirety herein by reference to the extent not inconsistent herewith.

Thus, compositions and methods against bacterial infections have been provided. In test studies against multiply drug-resistant bacteria, the compositions and methods demonstrated potent anti-microbial synergies of antibiotic and essential oil, achieving 100% kill rates at safe concentrations of composition components. Persons skilled in the art will appreciate that the present invention can be practiced by other than the described embodiments, which are presented for purposes of illustration rather than of limitation. The present invention is limited only by the claims that follow.

1. A composition of matter for application to a treatment site, the composition comprising:
an antibiotic first component, the antibiotic at a non-zero antibiotic concentration that when incubated, absent an essential oil second component, under standardized conditions with a population of viable pathogenic bacteria of at least about a million cells, leaves a non-zero population of the viable pathogenic bacteria, as per analytical assay; and
the essential oil second component, the essential oil at a non-zero essential oil concentration that, when incubated, absent the antibiotic first component, under the standardized conditions with a population of the viable pathogenic bacteria of at least about a million cells, leaves a non-zero population of the viable pathogenic bacteria, as per the analytical assay;
wherein:
the composition, with a combination of the first component at the antibiotic concentration and the second component at the essential oil concentration, incubated under the standardized conditions with a population of the viable pathogenic bacteria of at least about a million cells, leaves a population of zero of the viable pathogenic bacteria, as per the analytical assay.

2. The composition of claim 1 wherein the composition leaving a population of zero of the viable pathogenic bacteria indicates an antimicrobial synergy attained by the combination of the antibiotic and the essential oil at their respective concentrations, the combination exhibiting greater lethality toward the pathogenic bacteria than a sum of lethalities separately exhibited by either the antibiotic or the essential oil, at the respective concentrations.

3. The composition of claim 1 wherein the antibiotic includes an intercalating agent disruptive of bacterial DNA transcription or replication.

4. The composition of claim 1 wherein the antibiotic includes ethacridine lactate (EL) and the antibiotic concentration incubated with the populations of the viable pathogenic bacteria includes about 0.01%-1.0% (wt/wt) EL.

5. The composition of claim 1 wherein the antibiotic includes ethacridine lactate (EL) and the antibiotic concentration incubated with the populations of the viable pathogenic bacteria includes about 0.2% (wt/wt) EL.

6. The composition of claim 1 wherein the essential oil includes eugenol and the essential oil concentration incubated with the populations of the viable pathogenic bacteria includes about 0.05%-5.0% (vol/wt) eugenol.

7. The composition of claim 1 wherein the essential oil includes eugenol and the essential oil concentration incubated with the populations of the viable pathogenic bacteria includes about 0.3% (vol/wt) eugenol.

8. The composition of claim 1 wherein the essential oil includes tea tree oil and the essential oil concentration incubated with the populations of the viable pathogenic bacteria includes about 0.1%-10.0% (wt/wt) tea tree oil.

9. The composition of claim 1 wherein the essential oil includes tea tree oil and the essential oil concentration incubated with the populations of the viable pathogenic bacteria includes about 4.0% (wt/wt) tea tree oil.

10. The composition of claim 1 wherein the pathogenic bacteria include Gram positive methicillin-resistant Staphylococcus aureus (MRSA).

11. The composition of claim 1 wherein the pathogenic bacteria include Gram negative neuropathogenic Escherichia coli K1 (E. coli K1).

12. The composition of claim 1 wherein the pathogenic bacteria include Gram negative Pseudomonas aeruginosa(P. aeruginosa).

13. The composition of claim 1 wherein the essential oil is applied to the treatment site via an ointment base.

14. The composition of claim 1 wherein the combination of the essential oil and the antibiotic is applied to the treatment site via an ointment that includes the combination and an ointment base.

15. The composition of claim 14 wherein the ointment is embedded within a dressing, the dressing configured to transfer a dose of the combination to a subject dressed with the dressing.

16. The composition of claim 1 wherein the combination of the essential oil and the antibiotic is applied to the treatment site via a liquid.

17. A composition of matter for therapeutic application to a treatment site, the composition comprising:
an antibiotic first component, the antibiotic at a non-zero antibiotic concentration that, when incubated under standardized conditions, absent a second essential oil component, with a population of live Gram positive methicillin-resistant Staphylococcus aureus (MRSA) bacteria of at least about a million cells, leaves more than about 0.3% of the population viable, as determined by standard laboratory assay; and
the essential oil second component, the essential oil at a non-zero essential oil concentration that, when incubated under the standardized conditions, absent the first antibiotic component, with a population of live MRSA bacteria of at least about a million cells, leaves more than about 1% of the population viable, as determined by the standard laboratory assay;
wherein:
the composition, with a combination of the first component at the antibiotic concentration and the second component at the essential oil concentration, incubated under the standardized conditions with a population of live MRSA bacteria of at least about a million cells, leaves about 0% (zero) of the population viable, as determined by the standard laboratory assay, thereby demonstrating a synergistically elevated MRSA kill rate by the combined components of the composition over the sum of individual MRSA kill rates of the antibiotic first component and of the essential oil second component.

18. The composition of claim 17 wherein the antibiotic includes ethacridine lactate (EL) and the antibiotic concentration incubated with the populations of the live MRSA bacteria includes about 0.01%-1.0% (wt/wt) EL.

19. The composition of claim 17 wherein the essential oil includes eugenol and the essential oil concentration incubated with the populations of the live MRSA bacteria includes about 0.05%-5.0% (vol/wt) eugenol.

20. The composition of claim 17 wherein the essential oil includes tea tree oil and the essential oil concentration incubated with the populations of the live MRSA bacteria includes about 0.1%-10.0% (wt/wt) tea tree oil.

21. The composition of claim 17 wherein the combination of the essential oil and the antibiotic is applied to the treatment site, within an ointment that includes the combination and an ointment base.

22. The composition of claim 21 wherein the ointment is embedded within a suture material, the suture material configured to transfer a dose of the combination to a subject sutured with the suture material.

23. The composition of claim 17 wherein the combination of the essential oil and the antibiotic is applied in liquid form to the treatment site.

24. A composition of matter for application to a treatment site, the composition comprising:
an intercalating antibiotic first component, the intercalating antibiotic at a non-zero antibiotic concentration that when incubated, absent an essential oil second component, under standardized conditions with a population of viable pathogenic bacteria of at least about a million cells, leaves a non-zero population of the viable pathogenic bacteria, as per analytical assay; and
the essential oil second component, the essential oil at a non-zero essential oil concentration not exceeding about 75% (vol/wt), when incubated, absent the intercalating antibiotic first component, under the standardized conditions with a population of the viable pathogenic bacteria of at least about a million cells, leaves a non-zero population of the viable pathogenic bacteria, as per the analytical assay;
wherein:
the composition, with a combination of the first component at the antibiotic concentration and the second component at the essential oil concentration, incubated under the standardized conditions with a population of the viable pathogenic bacteria of at least about a million cells, leaves a population of zero of the viable pathogenic bacteria, as per the analytical assay.

25. The composition of claim 24 wherein the intercalating antibiotic disrupts bacterial cellular functions.

26. The composition of claim 24 wherein the intercalating antibiotic includes ethacridine lactate (EL) and the antibiotic concentration is applied, prior to incubation, to the populations of the viable pathogenic bacteria and includes about 0.01%-1.0% (wt/wt) EL.

27. The composition of claim 24 wherein the essential oil includes eugenol and the essential oil concentration is applied, prior to incubation, to the populations of the viable pathogenic bacteria and includes about 0.05%-5.0% (vol/wt) eugenol in an ointment base.

28. The composition of claim 24 wherein the essential oil includes tea tree oil and the essential oil concentration is applied, prior to incubation, to the populations of the viable pathogenic bacteria and includes about 0.1%-10.0% (wt/wt) tea tree oil in an ointment base.

29. The composition of claim 24 wherein the essential oil component is blended, prior to incubation with the pathogenic bacteria, with an ointment base including about 1% 15% (wt/wt) jojoba oil.

30. A composition of matter for therapeutic application to a treatment site against a MRSA infection, the composition comprising:
an antibiotic first component, the antibiotic at a non-zero antibiotic concentration that, when incubated under standardized conditions, absent a second essential oil component, with a population of live Gram positive methicillin-resistant Staphylococcus aureus (MRSA) bacteria of at least about a million cells, leaves more than about 3000 of the bacteria viable, as determined by standard laboratory assay; and
the essential oil second component, the essential oil at a non-zero essential oil concentration that, when incubated under the standardized conditions, absent the first antibiotic component, with a population of live MRSA bacteria of at least about a million cells, leaves more than about 1×10⁴ of the bacteria viable, as determined by the standard laboratory assay;
wherein:
the composition, with a combination of the first component at the antibiotic concentration and the second component at the essential oil concentration, incubated under the standardized conditions with a population of live MRSA bacteria of at least about a million cells, leaves about zero of the bacteria viable, as determined by the standard laboratory assay, thereby demonstrating a synergistically elevated MRSA kill rate by the combined components of the composition over the sum of individual MRSA kill rates of the antibiotic first component and of the essential oil second component; and
the composition is applied to the treatment site against the MRSA infection.
